(19) United States
(12) Patent Application Publication
(10) Pub. No.: US 2016/0291105 A1
(43) Pub. Date: Oct. 6, 2016

(54) MULTI-PARAMETRIC PET-MR IMAGING AND MULTI-MODALITY JOINT IMAGE RECONSTRUCTION

(71) Applicant: NEW YORK UNIVERSITY, New York, NY (US)

(72) Inventors: Florian Knoll, New York, NY (US); Martijn Anton Hendrik Cloos, New York, NY (US); Daniel K. Sodickson, New York, NY (US)

(73) Assignee: NEW YORK UNIVERSITY, NEW YORK, NY (US)

(21) Appl. No.: 15/051,450

(22) Filed: Feb. 23, 2016

Related U.S. Application Data
(60) Provisional application No. 62/120,322, filed on Feb. 24, 2015.
(60) Provisional application No. 62/120,667, filed on Feb. 25, 2015.

(51) Int. Cl. G01R 33/48; G01R 33/56; A61B 6/00; A61B 5/00; A61B 5/055; A61B 6/03

(52) U.S. Cl. CPC G01R 33/481; A61B 5/055; A61B 6/037; A61B 6/5205; A61B 5/742; A61B 6/461; A61B 6/5211; A61B 5/7225; A61B 5/7271; A61B 6/54; A61B 5/0042; A61B 6/501; G01R 33/5602

(57) ABSTRACT

A method of acquiring PET and MR images simultaneously includes obtaining raw k-space data for continuous MR volumes and acquiring PET information. The method further includes performing a joint multi-modality image reconstruction and generating a set of PET and MR images. The method additionally includes generating a set of MR fingerprints from the reconstructed MR images and using the MR fingerprints to generate a set of parameter maps.

### CROSS-REFERENCE TO RELATED PATENT APPLICATIONS

This application claims priority from Provisional Application U.S. Application 62/120,322, filed Feb. 24, 2015, incorporated herein by reference in its entirety. This application claims priority from Provisional Application U.S. Application 62/120,667, filed Feb. 25, 2015, incorporated herein by reference in its entirety.

### FIELD

The present invention generally relates to magnetic resonance (MR) soft tissue imaging and positron emission tomography (PET) functional imaging.

### BACKGROUND

PET-MR systems integrate PET and MR imaging techniques to capture soft tissue images as well as morphological and functional data. Current state-of-the-art PET-MR systems allow simultaneous acquisition of MR and PET data. Such systems enjoy the benefit of MR, which delivers a wide range of contrasts, high soft tissue contrast and high spatial resolution, and the benefit of functional quantitative information from PET.

### SUMMARY

Certain implementations of the present disclosure relate to apparatuses, methods, and computer-readable media with instructions thereon for carrying out simultaneous PET-MR imaging with substantially reduced acquisition times and/or substantially improved image quality, resulting in the generation of multiple quantitative MR and PET parameter maps from a single simultaneous acquisition.

According to an implementation, a method of acquiring PET and MR images simultaneously includes obtaining raw k-space data for continuous MR volumes and acquiring PET information. The method further includes performing a joint multi-modality image reconstruction and generating a set of PET and MR images. The method still further includes generating a set of MR fingerprints from the reconstructed MR images and using the MR fingerprints to generate a set of parameter maps.

Additional features, advantages, and implementations of the present disclosure are apparent from consideration of the following detailed description, drawings, and claims. Moreover, it is to be understood that both the foregoing summary of the present disclosure and the following detailed description are exemplary and intended to provide further explanation without limiting the scope of the present disclosure and the claims.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 illustrates an apparatus for PET-MR imaging according to an implementation;

FIG. 2 illustrates a computer system for PET-MR imaging according to an implementation;

FIG. 3 illustrates a process for PET-MR imaging according to an implementation;

FIG. 4 illustrates a conventional PET-MR acquisition protocol;

FIG. 5 illustrates a PET-MR data acquisition protocol according to an implementation;

FIG. 6 illustrates PET reconstructions and quantitative parameter maps according to an implementation;

FIG. 7 illustrates contrasts generated from quantitative maps shown in FIG. 6;

FIG. 8 illustrates a preprocessing operation according to an implementation;

FIG. 9 illustrates an acquisition protocol according to an implementation;

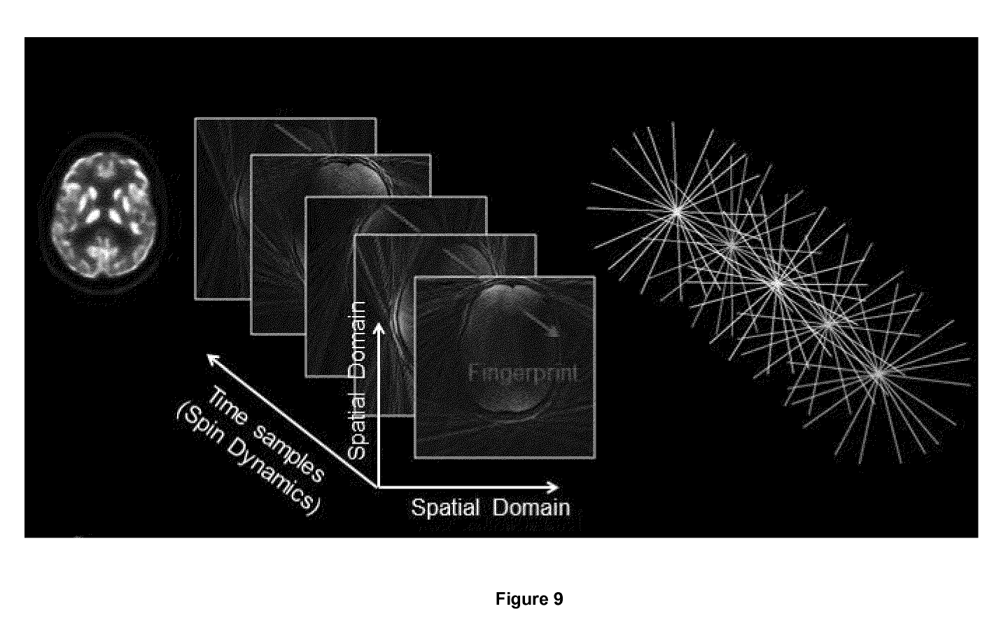

FIG. 10 illustrates results from a PET-MR acquisition according to an implementation; and

FIG. 11 illustrates pre-contrast and post-contrast results from a PET-MR acquisition according to an implementation.

### DETAILED DESCRIPTION

In the following detailed description, reference is made to the accompanying drawings, which form a part hereof. In the drawings, similar symbols typically identify similar components, unless context dictates otherwise. The illustrative implementations and/or embodiments described in the detailed description, drawings, and claims are not meant to be limiting. Other implementations and/or embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

Despite offering benefits as described above, PET-MR systems suffer from drawbacks which may make them ill-suited to routine clinical practice. One of the limiting factors in routine clinical use is the increased complexity and required scan time associated with the MR aspects of such systems.

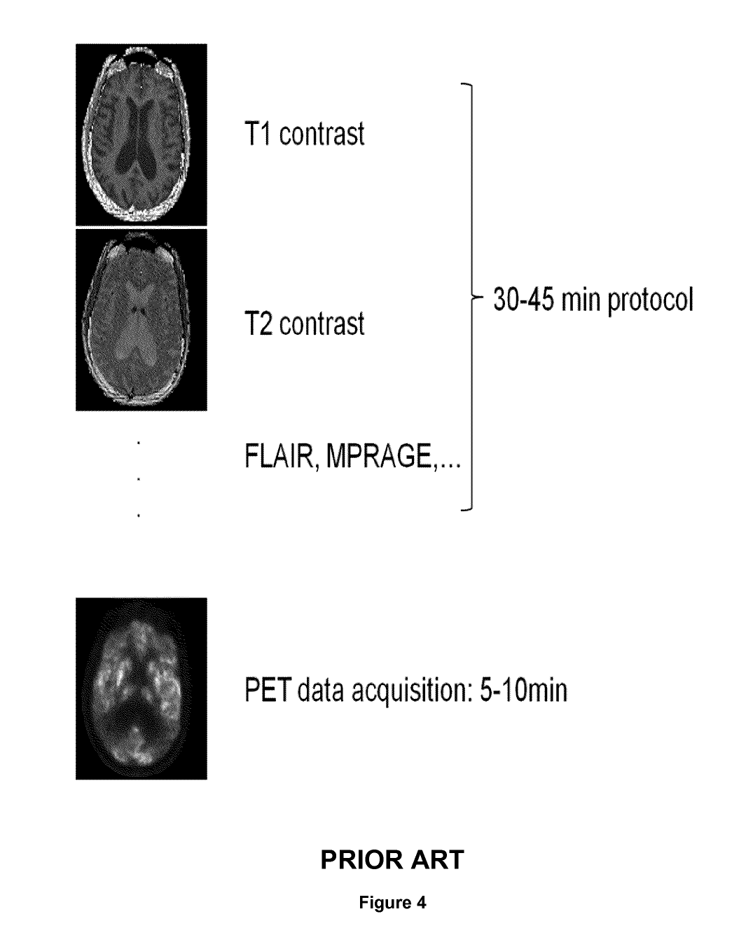

FIG. 4 illustrates a typical PET-MR acquisition paradigm for a conventional system. In particular, FIG. 4 illustrates an acquisition paradigm including an MR component followed by a PET component. The MR component may include, for example, a T₁ contrast, a T₂ contrast, as well as fluid-attenuated inversion recovery (FLAIR) and a magnetization prepared rapid gradient echo (MPRAGE) sequences. As shown in FIG. 4, the T₁ sequence, the T₂ sequence, and additional steps proceed sequentially.

As indicated in FIG. 4, it is possible to acquire PET data of diagnostic quality in 5 to 10 minutes per bed position. In contrast, as FIG. 4 indicates, collecting a clinically relevant variety of traditional MR contrasts may require a protocol of 30 to 45 minutes. Thus, for a case where PET data acquisition takes 5 minutes and MR contrast acquisition takes 45 minutes, 90% of the time is taken up by MR. Accordingly, in conventional PET-MR systems, the MR approach amounts to a bottleneck, and such systems suffer from relatively inefficient use of the PET component. Further, such systems require particularly long scan times for PET protocols involving multiple bed positions (5-10 minutes per bed position). Additionally, the PET component may acquire more data than is needed, further contributing to system inefficiency.

In the implementations described herein, continuous multi-parametric MR data acquisition may be carried out in tandem with PET acquisition. In certain implementations, a method of data acquisition includes acquiring PET and MR data in a simultaneous and continuous acquisition without exceeding conventional stand-alone PET acquisition times of 5-10 minutes per bed position. Moreover, in certain implementations, continuous MR data may be acquired such that all relevant contrast information is advantageously encoded simultaneously in a single imaging sequence. For example, the contrast information may be encoded simultaneously as described in U.S. Patent Application 61/904,716 to Cloos et al., entitled “Generalized signal encoding for magnetic resonance fingerprinting,” and filed on Nov. 15, 2013, the contents of which are hereby incorporated by reference in their entirety for the techniques and background information contained therein.

Additionally, in such implementations, RF-field non-uniformities are automatically filtered out, unlike conventional approaches. By automatically filtering out such non-uniformities, such implementations avoid inadvertent introduction of bias. In addition to these benefits, certain implementations allow for use in a parallel transmission setting.

Further, raw MR data acquired according to certain implementations may be reconstructed together with raw PET data. Such reconstruction is accomplished using a dedicated joint multi-contrast multi-modality image reconstruction framework that treats a plurality of contrasts and both the MR and PET modalities as a single imaging dataset. The resulting MR images may then be fitted, pixel-by-pixel or in other relevant groupings, to a database or repository of simulated MR signal time courses to derive quantitative parameters (e.g., T₁, T₂, PD, B₁⁺) which determine the MR contrast.

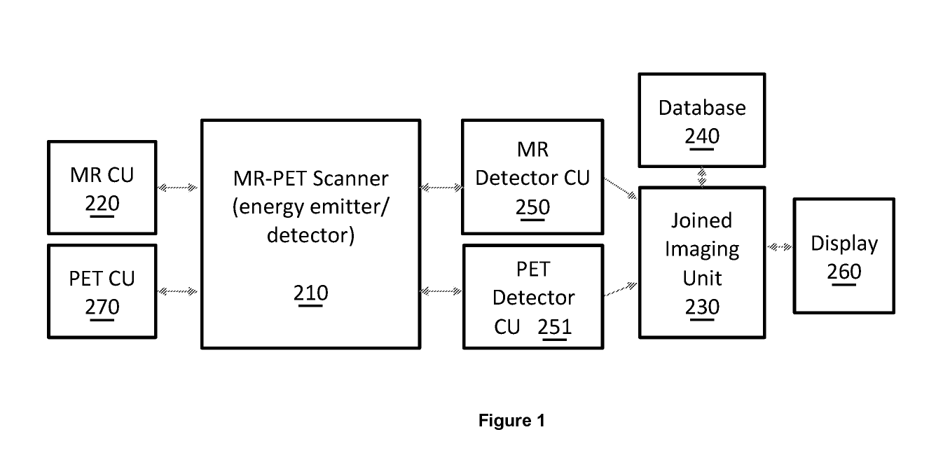

As mentioned above, at least one implementation is directed to an apparatus. In particular, at least one implementation relates to an apparatus for multi-parametric PET-MR and multi-modality joint image reconstruction. FIG. 1 depicts a system 200 comprising at least one component configured to carry out PET-MR and joint image reconstruction. As shown in FIG. 1, the system 200 includes a device 210 configured to apply energy to a volume of an object (a volume, for example, in a human or animal body), an MR control unit 220 and a PET control unit 270. The system 200 may optionally include an imaging unit 230, a display 260, and a database 240. A MR detector control unit 250 and a PET detector control unite 251 may be included.

Referring again to FIG. 1, the device 210 may be an energy emitter such as a magnetic resonance imaging apparatus that emits RF energy. The device 210 may, in some implementations, include a control unit containing control logic for controlling various aspects of the emission of energy from the emitter 210. In some implementations, the device 210 does not rely on a single control unit, and is instead controlled by the MR control unit 220 and/or the PET control unit 270. In one implementation, the control units 220, 270 are configured to communicate with the MR PET scanner 210 and a MR detector control unit 250 and a PET Detector control unit 251.

Again in reference to FIG. 1, each of the MR control unit 220 and the PET control unit 270 is a controller configured to communicate with the emitter 210 to control the application of energy to the volume. For example, the MR control unit 220 may be configured to command the emitter 210 to provide pulses of energy with fixed or variable characteristics.

Furthermore, each of the control units 220, 270 may cooperate with an imaging unit 230 to produce the imaging segments. In some instances, the imaging unit 230 may be integrated within the control unit 220 and/or the control unit 270, while in other implementations, the imaging unit 230 may be physically separated and distinct from the control unit 220 and/or the control unit 270. In some implementations, the imaging unit 230 may be coupled to a display 260 that may provide information about the imaging. The display 260 may allow, via touch-screen capability, manipulation of any combination of the control units 220, 270 and the imaging unit 230.

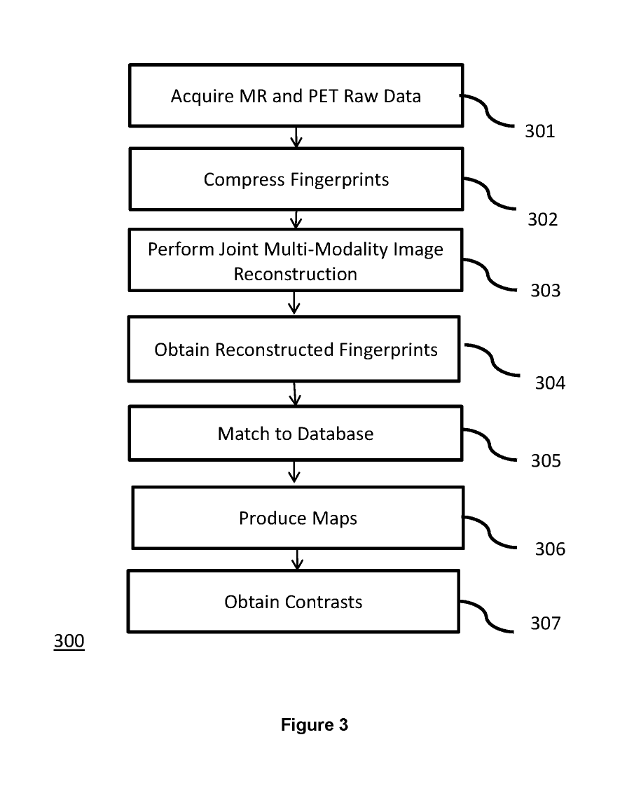

Additionally, each of the control units 220, 270 is configured such that at least one relaxation parameter is encoded into the single continuous imaging segment (i.e., a fingerprint). For example, both T₁ and T₂ relaxation parameters may be encoded. The control units 220, 270 may be configured to carry out the operations shown in FIG. 3 so as to complete acquisition within clinically acceptable scan times for a variety of scans (e.g., cartilage scans, brain scans).

Referring once more to FIG. 1, the control unit 220 and/or the control unit 270 may be configured to communicate with a database 240. The database 240 may be in an external computing device (not shown) or may be integrated within one or both of the control units 220, 270 or the imaging unit 230. The database 240 may store data relating to each acquired fingerprint. The database 240 may facilitate comparison of data acquired in a scan to entries in the database 240. In some implementations, the database 240 may be remotely connected to one or both of the control units 220, 270. The database 240 allows the reconstructed fingerprints to be matched, as described above.

Further, certain implementations, such as the implementation of FIG. 1, are configured to produce a set of quantitative MR parametric maps and PET images. The PET images benefit from higher spatial resolutions of the MR data via joint reconstruction, in which shared features arising from common underlying anatomy in the MR and PET scans are emphasized. Thus, a point spread function of reduced width (corresponding to improved spatial resolution) is achieved in comparison to typical PET reconstruction. Additionally, required MR contrasts may be generated retrospectively from the MR parametric maps using signal equations which are associated with the MR-sequences used in various clinical protocols. For example, if quantitative T₁ and T₂ maps are generated in an imaged subject, it is possible to simulate what a T₁-weighted image, or a T₂-weighted image, or an image with mixed T₁ and T₂ weighting, would have looked like in that subject. This process of weighted image synthesis is referred to as “retrospective contrast generation” or “production of synthetic contrasts.”

FIG. 3 illustrates a PET-MR data acquisition protocol according to at least one implementation. As indicated above, the protocol includes, inter alia, obtaining raw k-space data and raw PET data (301). The protocol may further include performing fingerprint compression (302), which may be omitted in some implementations. The protocol still further includes performing a joint multi-modality image reconstruction (303). The reconstruction yields a set of PET images, a set of MR images, and at least one set of reconstructed fingerprints based on the MR images (304). In an alternative embodiment, the image reconstruction may occur prior to compression.

Referring again to FIG. 3, the protocol further includes performing database matching of at least one set of reconstructed fingerprints (305). More specifically, the fingerprints are matched to a database of signal evolutions to estimate MR parametric maps, as discussed further below (306). Additionally, the protocol still further includes performing retrospective contrast generation and producing synthetic contrasts (307).

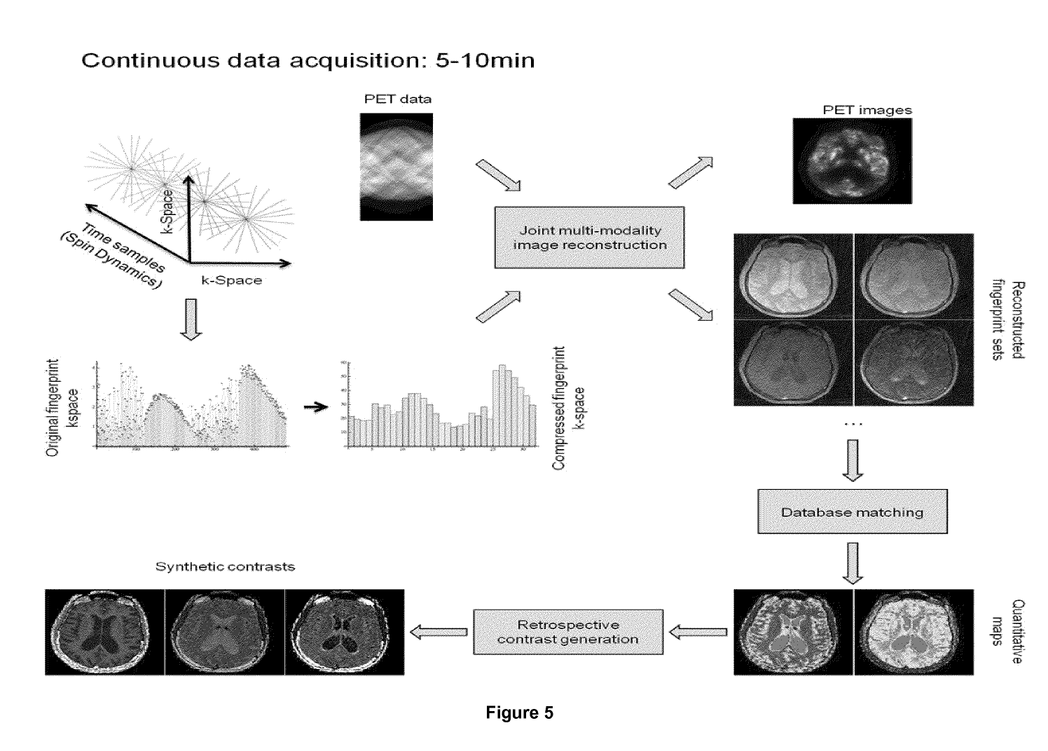

FIG. 5 illustrates various operations in a PET-MR data acquisition protocol according to the implementation described above, as well as the results of such operations. As shown in FIG. 5, multiple PET and MR images may be produced, by way of illustration, as a result of the joint multi-modality image reconstruction, and fingerprints may be derived from the reconstructed MR images. As is also shown in FIG. 5, the reconstruction may follow after compression. In some implementations, these fingerprints may further be compressed, for example by averaging in time. Further, a plurality of quantitative maps may be produced following the database matching of the derived fingerprints. As indicated in FIG. 5, performing the retrospective contrast generation based on the quantitative maps yields a plurality of differing synthetic contrasts.

According to certain implementations, MR and PET data are acquired simultaneously, such that there is a negligible idle time or no idle time whatsoever of the PET and MR components of an exemplary PET-MR system. The scan time of the MR component may be advantageously reduced to 5-10 minutes, i.e., far shorter than conventional MR systems. Owing to the simultaneous acquisition, the total scan time may therefore be confined to being within 5-10 minutes. Even though the scan time is substantially reduced, the required differing contrasts for a diagnostic clinical protocol are still attained. Moreover, the approach of various embodiments may further yield quantitative parameter maps of T₁, T₂, relative proton density (PD), and transmit coil sensitivity B₁⁺, for example. If additional sequences were added to a typical MR acquisition protocol to obtain such maps, the total data acquisition time would be prolonged considerably, so as to be infeasible in many instances for clinical settings. In other words, compared to conventional approaches, a wealth of data may be obtained in an extremely efficient manner, well within clinically acceptable scan times.

Furthermore, various implementations described herein achieve images of superior quality. Such implementations yield higher quality images than those of conventional MRF. MRF techniques are robust against incoherent undersampling artifacts only to a certain threshold, beyond which image quality is degraded. In certain implementations, the joint multi-modality reconstruction does not rely purely on incoherence between undersampling artifacts and simulated signal evolutions. Rather, a non-linear joint multi-modality reconstruction simultaneously reconstructs a series of MRF images and a PET image by enforcing joint sparsity. In other words, it is assumed that, though the MR and the PET images differ substantially in content and contrast, these differences are limited in number (i.e. sparse) in an appropriate domain, since both sets of simultaneously acquired images arise from a common underlying anatomy. By controlling joint sparsity, the presence of residual undersampling artifacts in the MR component is beneficially reduced. For example, streaking artifacts resulting from MR data undersampling may be effectively eliminated, since these artifacts are not consistent between the MR and the PET images. At the same time, controlling joint sparsity improves the quality of the PET reconstruction. Moreover, through such an enforcement mechanism, data consistency in both the PET and MR modalities may be ensured. The joint MR-PET reconstruction may be performed by minimizing the function listed below as Equation 1:

$${{arg min}_{{x}_{MR} , {x}_{PET}} \{ {‖ E ( {x}_{MR} ) - k ‖}_{2}^{2}}_{First Term} {+ {\sum }_{j = 1}^{J} ( {( A ( {x}_{PET} ) )}_{j} - {f}_{j} {log ( A ( {x}_{PET} ) )}_{j} )}_{Second Term} + {λ {‖ {‖ \begin{matrix}Ψ ( {x}_{MR} ) \\ Ψ ( {x}_{PET} )\end{matrix} ‖}_{2} ‖}_{1} \}}_{Third Term} . \qquad Equation 1$$

In Equation 1 shown above, the first term corresponds to the data consistency of the MR data according to a least-squares approach. The second term is the PET data consistency (expectation-maximization), and the rightmost term is a control that enforces joint sparsity between MRF and PET. Specifically, xMR is the series of 3D MRF image volumes, k is the series of undersampled MR k-space datasets, and E is used to map xMR to k and accounts for coil sensitivity modulations. Further, A is a PET projection operator mapping the image xPET to sinogram (i.e., ordered projection) data f. Additionally, j corresponds to indices for the PET lines of response, while J is the total number of the PET lines of response (i.e., measured photon coincidence counts that localize to particular rays passing through the imaged subject). Also, λ is a regularization parameter and ψ is a sparsifying transform. The sparsifying transform accounts for both the PET and MR components.

After performing reconstruction in accordance with Equation 1, the resulting series of MRF images are then matched to the database of signal evolutions to estimate MR parametric maps, as indicated in FIG. 3 (305). Further, by performing reconstruction according to Equation 1, particular advantages may be realized by the various implementations described herein in comparison to conventional approaches. Moreover, certain implementations facilitate post-examination PET-driven virtual MR examination, e.g., by first using PET to identify suspicious hotspots, and then synthesizing desired MR contrasts for the hotspots retrospectively during the process of reading the images, in order to simulate how those hotspots might have appeared in a range of diagnostic MR scans that a physician might have wished to order based on the PET results.

In particular, due to extremely high undersampling factors, even when including a contribution from the PET component, image reconstruction may be challenging. An MR fingerprint x_{MR} may contain a large number of time samples (480 time samples, for example). Ascertaining the values of multiple parameters (e.g., five parameters) entails solving a highly over determined problem. However, by integrating fingerprints along a time dimension in the complex image space prior to matching, the conditioning may be advantageously improved. In the joint reconstruction according to various implementations, samples are integrated over time in bins of chosen width.

Performing compression in pre-processing improves conditioning of the image reconstruction. In one exemplary implementation, as illustrated in FIG. 5, the image reconstruction may form a compressed fingerprint of just 32 samples, for example, each of which accumulates spin dynamics variations of 15 different samples. Moreover, performing compression as a pre-processing operation prior to database matching may accelerate the pace of the reconstruction.

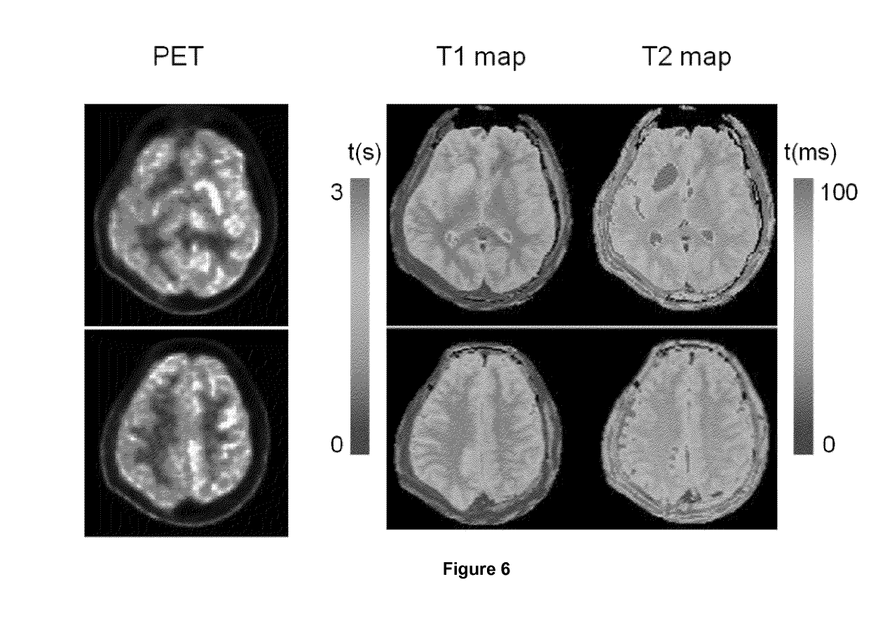

FIG. 6 illustrates PET reconstructions and quantitative parameter maps of a representative examination according to an implementation. The data shown in FIG. 6 were acquiring using a 3T PET-MR system produced by Biograph mMR, Siemens, Erlangen, Germany. The acquisition time for the images shown in FIG. 6 totaled 6 minutes, indicating that acquisition may be completed efficiently within the PET timeframe of 5-10 minutes. The scanning was carried out using a 12 channel head coil, an injection of 10 mCi 18 F-fludeoxyglucode (FDG) with an uptake time of 45 minutes, a 19 s Dixon AC scan, 6 minutes of continuously acquired data fingerprints, 30 slices with a slice thickness of 3.5 mm, an in-plane resolution of 1.5 mm×1.55 mm, a 176×176 matrix, and 480 sets each having 5 radial spokes.

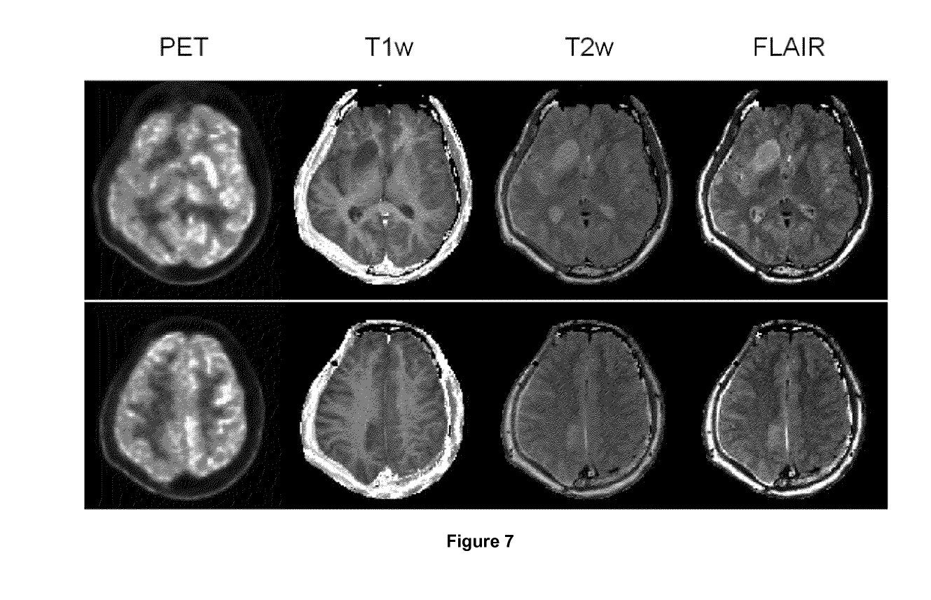

A quantitative analysis of the jointly reconstructed PET images in comparison to an ordered subset expectation maximization (OSEM) showed a reduction of 6% PET signal in areas of cerebrospinal fluid flow (CSF) where no FDG uptake was expected, indicating a reduction of the partial volume effect. In gray matter and white matter, the differences were less prominent (GM: 1% signal increase, WM: 1% signal decrease with joint reconstruction). FIG. 7 illustrates retrospective T₁ weighted, T₂ weighted, and FLAIR contrasts generated from the quantitative maps shown in FIG. 6.

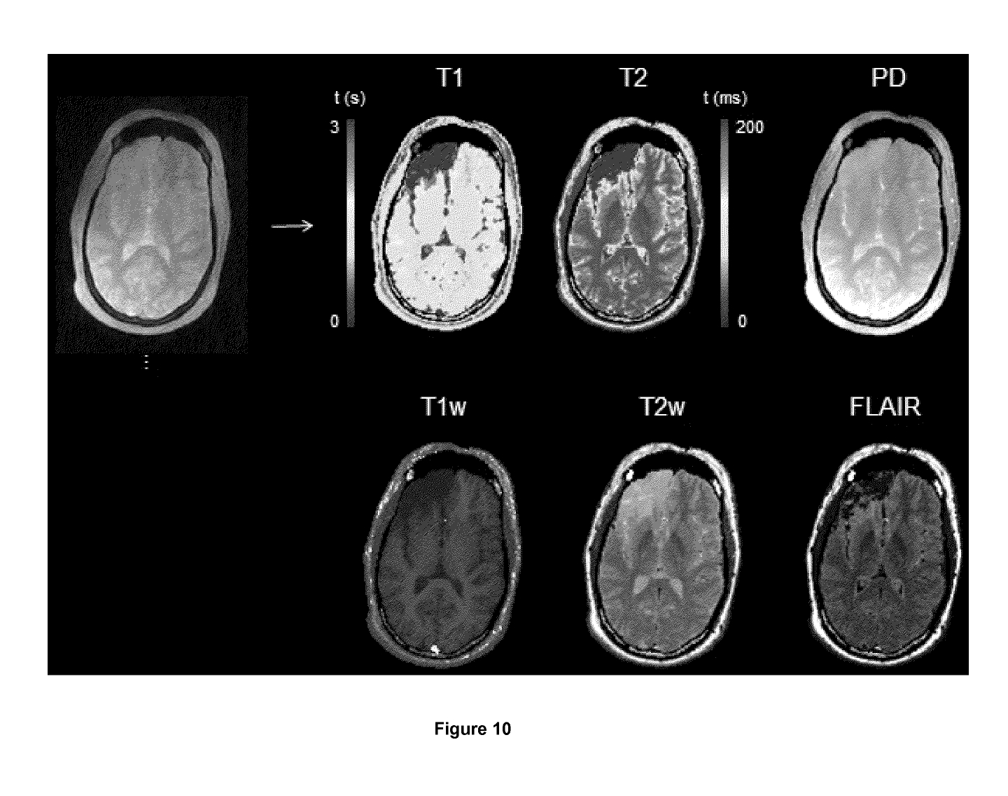

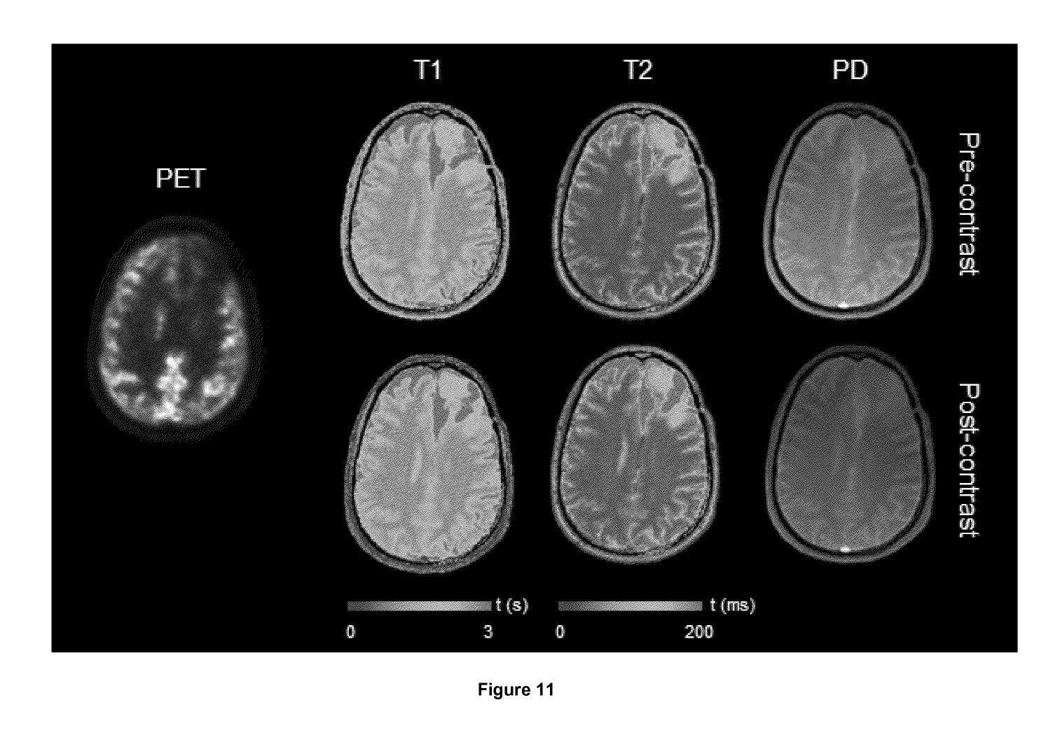

FIG. 10 depicts MR results from a PET-MR acquisition in the brain of a patient with epilepsy according to an implementation. Illustrated in FIG. 10 are an example source MR image, quantitative T₁ and T₂ maps, a relative proton density map, and images with synthesized T₁, T₂, and FLAIR contrast. FIG. 11 illustrates pre-contrast and post-contrast results from a PET-MR acquisition in a patient with a brain tumor according to an implementation FIG. 11 contains the jointly reconstructed PET image along with quantitative MR parameter maps pre and post contrast.

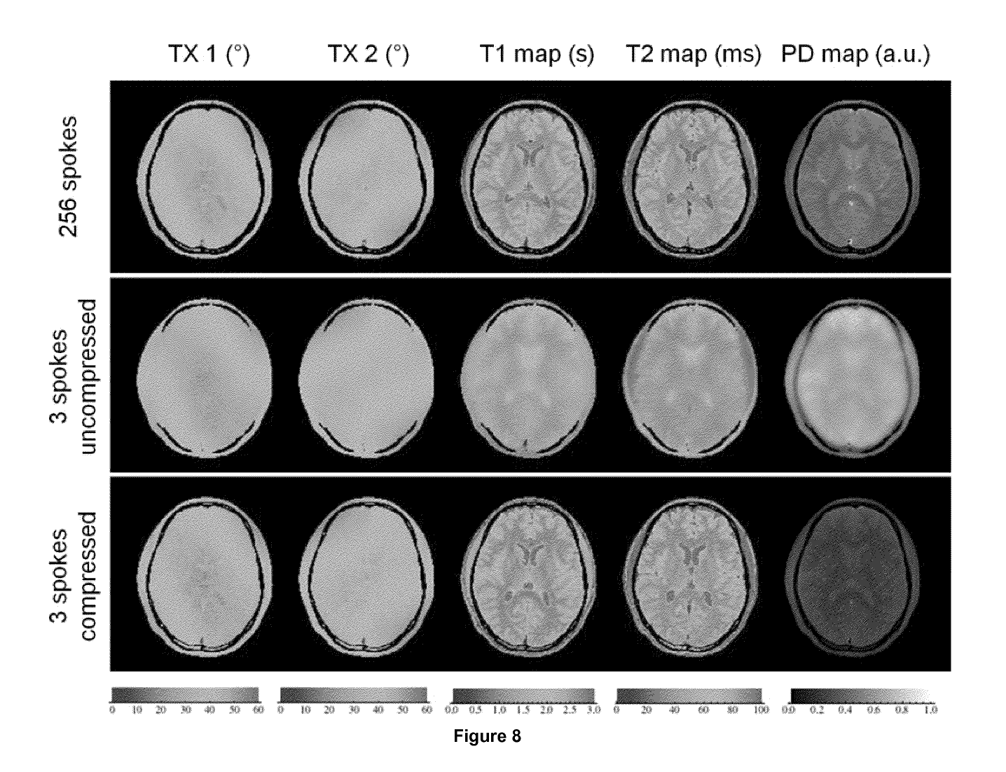

As mentioned above, certain implementations employ MR fingerprinting techniques, namely in encoding contrast information in a single imaging sequence. As discussed above, such implementations may employ fingerprint compression techniques as a pre-processing measure prior to database matching. FIG. 8 illustrates the results of a preprocessing operation according to an implementation, in which fingerprint compression is carried out. The top row of fingerprints in FIG. 8 illustrates maps generated from a full sampling of 256 radial spokes. The middle row shows maps generated from a highly undersampled data with only 3 spokes, without compression, while the bottom row shows maps generated from 3 spokes with compression.

Furthermore, in certain implementations, such as those described above, reconstruction occurs iteratively. Iterative reconstruction differs from techniques in which compression takes place in image-space after reconstruction. Specifically, iterative reconstruction according to various implementations involves compression on raw k-space data, which compression is performed as a pre-processing operation prior to image reconstruction. Such an approach improves the condition of the pre-processing step.

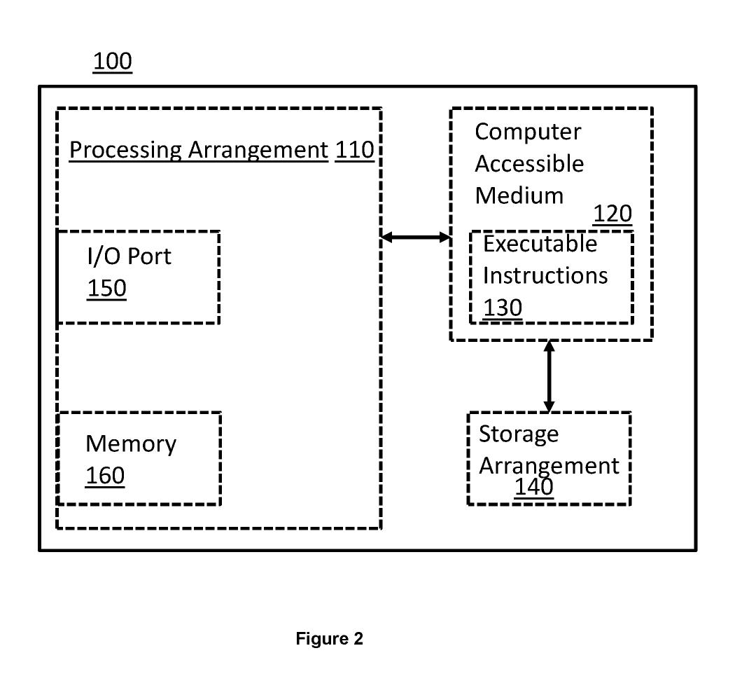

One embodiment of the invention relates to a system for magnetic resonance fingerprinting comprising a processor and a tangible computer-readable medium operatively connected to the processor. As shown in FIG. 2, e.g., a computer-accessible medium 120 (e.g., as described herein, a storage device such as a hard disk, floppy disk, memory stick, CD-ROM, RAM, ROM, etc., or a collection thereof) can be provided (e.g., in communication with the processing arrangement 110). The computer-accessible medium 120 may be a non-transitory computer-accessible medium. The computer-accessible medium 120 can contain executable instructions 130 thereon. In addition or alternatively, a storage arrangement 140 can be provided separately from the computer-accessible medium 120, which can provide the instructions to the processing arrangement 110 so as to configure the processing arrangement to execute certain exemplary procedures, processes and methods, as described herein, for example.

The instructions may include multiple of sets of instructions. For example, in some implementations, instructions are provided for acquiring k-space raw data, performing joint multi-modal image reconstruction, obtaining reconstructed fingerprints, compressing fingerprints, matching to a database such as the database 240, producing maps as shown in FIG. 6, and obtaining contrasts, for example, the contrasts shown in FIG. 7. In some implementations, instructions for compressing fingerprints may not be provided.

System 100 may also include a display or output device, an input device such as a key-board, mouse, touch screen or other input device, and may be connected to additional systems via a logical network. Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

Certain implementations described above achieve various advantages, including substantially reduced PET-MR acquisition times, as noted above. Further, in contrast to conventional approaches, certain implementations perform MR data acquisition continuously using a radial scheme that encodes all contrast information in one single imaging sequence, as indicated in FIG. 11. More particularly, as shown in FIG. 11, continuous radial scanning may be performed during PET acquisition. The radial scanning may be performed according to the MR fingerprinting techniques described above.

As noted above, such a process does not exceed the scan time of a clinical PET data acquisition. In particular, certain implementations complete the process within approximately 6 minutes. Further, by treating multiple MR contrasts and PET data as a single dataset during image reconstruction, correlations of the underlying anatomy of these datasets may be leveraged. Such an approach leads to an improved point spread function (PSF) for PET as well as improved removal of aliasing artifacts due to the high undersampling of the individual sets of MR images. Certain embodiments allow for quantitative MR parametric maps to be constructed from acquired MR data using pixel by pixel fitting to a database of simulated MR signal time courses, as indicated above.

Certain implementations may be used to provide PET-MR within a clinical setting and to encompass routine whole body and multiple bed position PET-MR screening on the same time scale as in PET-CT, while providing a full range of disparate MR contrasts and associated biological information content. Such implementations may be particularly conducive for tumor metastases screening, among other applications.

Further, such implementations provide for a significantly accelerated PET-MR protocol in which various operations are carried out simultaneously, in contrast to sequential protocols with prolonged acquisition times. Such a protocol may be efficiently executed to obtain PET image reconstruction with improved signal to noise ratios (SNR) and point spread functions.

Further, certain implementations facilitate ready access to quantitative MR information and may allow for numerous different contrasts to be generated retrospectively. Additionally, reduced MR examination time may permit whole body PET-MR screening. For typical 5-7 min PET scans, the time savings may be used to increase the number of MRF slices to provide larger volumetric coverage or increased slice resolution. In addition, the protocol allows a post-examination PET-driven virtual MR examination, in which PET may be used to identify suspicious hotspots, and the desired MR contrast for the target region may then be obtained retrospectively during the process of reading the images. Moreover, various implementations provide for improved and simplified tissue segmentation based on the generated T₁ and T₂ maps, due to the lack of spatial signal intensity modulation from transmit (B₁⁺) and receive coil sensitivity profiles.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. Therefore, the above embodiments should not be taken as limiting the scope of the invention.

What is claimed is:

1. A method of acquiring PET and MR images simultaneously, comprising:
obtaining raw k-space data for continuous MR volumes,
acquiring PET information
performing a joint multi-modality image reconstruction, generating a set of PET and MR images;
generating a set of MR fingerprints from the reconstructed MR images;
using the MR fingerprints to generate a set of parameter maps.

2. The method of claim 1, further comprising:
compressing the at least one set of reconstructed fingerprints.

3. The method of claim 1, further comprising:
comparing the at least one set of reconstructed fingerprints to a plurality of signal evolutions.

4. The method of claim 2, further comprising:
obtaining quantitative MR maps after comparing the at least one set of reconstructed fingerprints to the plurality of signal evolutions, and
performing retrospective contrast generation.

5. The method of claim 1, further comprising:
producing synthetic contrasts, including at least one of a T₁ contrast, T₂ contrast, a FLAIR contrast and an MPRAGE contrast.

6. The method of claim 1, wherein:
the PET and MR images are acquired within 5-10 minutes.

7. The method of claim 1, further comprising:
automatically filtering RF-field non-uniformities.

8. The method of claim 2, wherein each of the MR fingerprints is fitted pixel-by-pixel to respective signal evolution of the plurality of signal evolutions.

9. A computer implemented system for multi-parametric PET-MR IMAGING and multi-modality joint image reconstruction comprising:
a PET-MR scanner;
a MR control unit in communication with the PET-MR scanner;
a PET control unit in communication with the PET-MR scanner;
non-transitory computer-readable memory in communication with the PET-MR scanner having instructions stored therein for:
obtaining raw k-space data for continuous MR volumes, and
acquiring PET information and performing a joint multi-modality image reconstruction to generate a set of PET images and at least one set of reconstructed MR fingerprints.

10. The system of claim 9, further wherein the memory includes instructions for using the MR fingerprints to generate a set of parameter maps.

11. The system of claim 9, further comprising:
compressing the at least one set of reconstructed fingerprints.

12. The system of claim 9, further wherein the memory includes instructions for comparing the at least one set of reconstructed fingerprints to a plurality of signal evolutions.

13. The system of claim 10, further wherein the memory includes instructions for obtaining quantitative MR maps after comparing the at least one set of reconstructed fingerprints to the plurality of signal evolutions, and
performing retrospective contrast generation.

14. The system of claim 9, further wherein the memory includes instructions for producing synthetic contrasts, including at least one of a T₁ contrast, T₂ contrast, a FLAIR contrast and an MPRAGE contrast.

15. The system of claim 9, wherein:
the PET and MR images are acquired within 5-10 minutes.

16. The system of claim 9, further wherein the memory includes instructions for automatically filtering RF-field non-uniformities.

17. The system of claim 10, wherein each of the MR fingerprints is fitted pixel-by-pixel to respective signal evolution of the plurality of signal evolutions.

18. A method of simultaneously performing PET and MR imaging, comprising:
obtaining raw PET and MR data, including a plurality of 3D MRF image volumes, and
reconstructing PET and MR images;
wherein E is a mapping parameter, X_{MR} is the plurality of 3D MRF image volumes, k is the number of undersampled MR k-space datasets, J is a total number of PET lines of response, j are indices corresponding to the PET lines of response, f is sinogram data, A is a PET projection operator, x_{PET} is a PET image, λ is a regularization parameter, and ψ is a sparsifying transform.

19. The method of claim 18, wherein the PET and MR images are reconstructed in accordance with the following equation:
$${arg min}_{{x}_{MR} , {x}_{PET}} \{ {‖ E ( {x}_{MR} ) - k ‖}_{2}^{2} + {\sum }_{j = 1}^{J} ( {( A ( {x}_{PET} ) )}_{j} - {f}_{j} {log ( A ( {x}_{PET} ) )}_{j} ) + λ {‖ {‖ \begin{matrix}Ψ ( {x}_{MR} ) \\ Ψ ( {x}_{PET} )\end{matrix} ‖}_{2} ‖}_{1} \} .$$
